(19) United States
(12) Patent Application Publication
(10) Pub. No.: US 2005/0181364 A1
(43) Pub. Date: Aug. 18, 2005

(54) Assay

(76) Inventor: Frank Frantzen, Oslo (NO)

Correspondence Address: NEEDLE & ROSENBERG, P.C., SUITE 1000, 999 PEACHTREE STREET, ATLANTA, GA, 30309-3915, US

(21) Appl. No.: 10/502,226

(22) PCT Filed: Feb. 6, 2003
(86) PCT No.: PCT/GB03/00529

(30) Foreign Application Priority Data
Feb. 6, 2002 (GB) 0202776.1

(51) Int. Cl. C12Q 1/68; G01N 33/53; G01N 33/537; G01N 33/543

(52) U.S. Cl. 435/6; 435/7.92; 436/86

(57) ABSTRACT

The present invention relates to a method of assaying for folate in a folate containing sample, wherein at least some of the folate comprises at least one attached glutamate residue. The method comprises subjecting the sample to hydrolysis to release paraminobenzoic acid, p-aminobenzoyl glutamic acid, or a salt thereof; contacting the released paraminobenzoic acid, p-aminobenzoyl glutamic acid, salt or a diazo derivative thereof, with a binding partner thereof; and directly or indirectly detecting the resulting binding partner: paraminobenzoic acid, binding partner: p-aminobenzoyl glutamic acid, or salt or derivative combination. The invention further relates to a kit usable in the method of the invention.

This invention relates to an assay for folate as well as to kits and apparatus therefor.

Folate, a term used to cover folic acid, dihydrofolate, tetrahydrofolate and methyltetrahydrofolate, is a coenzyme for the synthesis of thymidine monophosphate and thus of DNA, and is often referred to as being part of the vitamin B complex. It has been widely reported that low levels of folate or a disturbance in folate metabolism is involved in the pathogenesis of several disease states including in particular megaloblastic anaemia, neural tube defects, cancers and cardiovascular disease. Folate shortage in maternity has also been linked to childhood leukaemia. Accordingly the accurate assessment of folate is clinically important in disease and risk evaluation and monitoring.

Assessment of folate however is complicated by the fact that it occurs in a multiplicity of forms varying for example in pteridine ring structure and the number of glutamate residues and in the distribution of the different variants as the distribution is different for folate in serum and in erythrocytes. Indeed there are currently two widely used folate assays—one for serum and the other for whole blood. However for whole blood folate there are large variances in the interassay and intraassay results from different laboratories. As erythrocyte folate gives a measure of the long term status of the patient and is less influenced by food intake than is serum folate, it is generally preferred to assay for erythrocyte folate; however in view of the problems with reliably assaying erythrocyte folate some laboratories are proposing to revert to measuring serum folate instead.

It has been proposed to eliminate the problems arising from the heterogenicity of folate by subjecting the assay sample to prolonged boiling (about six hours) in half-concentrated hydrochloric acid followed by chromatographic separation and subsequent assessment (e.g. by mass spectroscopy) of a folate fragment that is common to all variants, namely para-amino benzoic acid (PABA). See for example Anal. Biochem. 283: 266-275 (2000). The effect of this harsh treatment and separation is to cleave the pteridine ring and strip off all of the attached glycine residues from all variants of folate to leave PABA. Such a technique however is not feasible for routine clinical use, e.g. in diagnostic laboratories or at the point of care. This is particularly due to the nature of the chromatographic separation step, which is time consuming and ill suited to high throughput or multiple-parallel analysis.

There is thus an ongoing need for a reliable and viable assay for folate in biological specimens.

We have now found that such an assay may be based on detection of PABA without requiring its chromatographic separation from the sample and without requiring harsh and user-unfriendly or impractical reaction conditions.

Thus viewed from one aspect the invention provides a method of assaying for folate in a folate containing sample, said method comprising:
subjecting said sample to hydrolysis to release paraminobenzoic acid, p-aminobenzoyl glutamic acid (PABA-glu), or a salt thereof; contacting the released paraminobenzoic acid, PABA-glu or salt, or a diazo derivative thereof, with a binding partner therefor; and directly or indirectly detecting the resulting binding partner:paraminobenzoic acid, binding partner:PABA-glu, or salt or derivative combination.

Preferably the invention provides a method of assaying for folate in a folate containing sample, said method comprising:

subjecting said sample to hydrolysis to release paraminobenzoic acid or a salt thereof; contacting the released paraminobenzoic acid or salt or a diazo derivative thereof with a binding partner therefor; and directly or indirectly detecting the resulting binding partner:paraminobenzoic acid or salt or derivative combination.

The sample assayed according to the method of the invention may be any folate-containing sample, but especially preferably is blood or derived from blood, e.g. concentrated red blood cells or serum, more especially concentrated (and if desired washed) red blood cells (RBC). With an RBC sample, if desired the cells may be lysed and protein therefrom denatured before the hydrolysis; optionally and preferably however the hydrolysis treatment itself will cause cell lysis and protein denaturing.

In the assay method of the invention, the binding partner may be in solution or it may be immobilized on a macrostructure, e.g. a solid, liquid or gel particle, or a substrate surface, for example a sheet, rod, tube, fibre, mesh, web, etc. The binding partner:PABA, binding partner:PABA-glu, etc combination may be directly detectable, e.g. by virtue of characteristic radiation emission or absorption, or enzymatic activity. Alternatively it may be indirectly detectable, e.g. by virtue of the ability of the binding partner to bind to a competitive substance which produces a directly detectable combination with the binding partner.

In one preferred aspect, the binding partner is an antibody or antibody analog capable of binding PABA, PABA-glu, or a PABA derivative, etc, e.g. an antibody, an antibody fragment, a single chain antibody or antibody fragment, an oligopeptide, an oligonucleotide or a small organic molecule. Using an appropriate antigen, e.g. PABA, PABA-glu, or a PABA (or PABA-glu) conjugate or derivative, such binding partners may be selected using conventional techniques, e.g. in vivo antibody generation, library techniques such as phage display, combinatorial chemical techniques and computer-aided molecular design.

In a particularly preferred embodiment however the binding partner is an aromatic tertiary amine or phenol or phenol derivative capable of coupling to para diazo benzoic acid (PDBA) or paradiazobenzoyl glutamic acid (PDBA) to form a diazo compound having a characteristic light absorption or emission. (Light used here includes radiation outside the visible wavelength range, e.g IR and UV, in particular near IR).

In this embodiment, the released PABA or PABA-glu is converted to PDBA, or PDBA-glu respectively e.g. by reaction with nitrite, preferably before being contacted with the binding partner.

Where antibody generation is to be used to select the binding partner, it is preferred to use as the antigen a PABA or PABA-glu substituted at the 2 or 3 position by a group covalently bound to an antigenic macromolecular carrier, e.g. a protein such as tetanus toxoid, keyhole limpet hemocyanin (KLH), bovine serum albumin (BSA), or other proteins commonly used for this purpose. One preferred starting point for producing such antigens is hydroxy and/or nitro substituted PABA or PABA-glu derivatives, such as for example 2-hydroxy-4-amino-benzoic acid, 2-hydroxy-4-nitro-benzoic acid, 3-hydroxy-4-amino-benzoic acid, 3-hydroxy-4-nitro-benzoic acid, or their monoglutamyl amides etc. The 2-hydroxy group may readily be reacted with a coupling agent, preferably after activation, e.g. tosylation. During the coupling reaction, the 4-amino group (and in some cases also the carboxyl group or groups) may desirably be protected. Conventional protecting groups, e.g. Fmoc, Boc or acetamide for the amine group, and ester formation for the carboxylic acid group or groups, may be used in this regard.

Alternatively, coupling groups or functional groups reactive with coupling agents may be introduced at the 2 or 3 positions of PABA or PABA-glu using conventional chemical techniques. Where it is desired to introduce a group at the 2 position, it may be preferred to use para nitro benzoic acid or the monoglutamyl amide thereof and to reduce the nitro group to an amine group (e.g. with Li/H₂) following introduction of the 2-substituent.

The group linking the PABA or PABA-glu to the carrier preferably provides a 1 to 50 atom bridge linking the two, more preferably a 5 to 20 atom bridge. The backbone atoms of the bridge may or may not be part of an inflexible structure (e.g. an aromatic ring or an aliphatic cage), e.g. to secure correct orientation of the PABA or PABA-glu residue.

Coupling of the 2 or 3 substituted PABA or PABA-glu to the carrier may be effected in conventional manner, e.g. using thiol terminated 2/3-substituents (for example terminated with a Cys residue) and disulphide or maleimide functionalized carriers.

Less preferably, the antigen for raising anti-PABA or anti-PABA-glu antibodies may be prepared by coupling an antigenic macromolecular carrier (e.g. a protein) to the carboxy or amino functions of PABA itself or to the amino or one of the carboxy groups of PABA-glu. In the case of the coupling to the amino function, a Fmoc protected NHS-activated amino alkanoic acid (e.g. amino propanoic acid) spacer may be reacted with PABA or PABA-glu, de-protected then coupled to a carrier protein. Alternatively, a protein coupled to an aldehyde-group carrying spacer may be condensed with the amino group of PABA or PABA-glu to yield an antigen. A further alternative is to react an isothiocyanate activated aminoalkanoic acid spacer with PABA or PABA-glu or to react isothiocyanate-activated PABA or PABA-glu with an amino alkanoic acid spacer, optionally after first coupling the spacer to a carrier protein.

The binding partner is preferably substrate bound, e.g. to a porous web (e.g. nitrocellulose) or to polymeric beads, especially preferably magnetically collectable beads (e.g. such as those available from Dynal AS, Oslo, Norway). With an immobilized binding partner, it is possible to wash the substrate after it has been incubated with the PABA or PABA-glu solution and thus remove other materials in the sample that might interfere with determination of the PABA:binding partner or PABA-glu:binding partner combination. This is particularly desirable where the sample contains haemoglobin and the binding partner forms an azo compound on reaction with PDBA or PDBA-glu. Standard techniques for coupling the binding partner to the substrate can be used.

In the performance of the assay of the invention, the conversion of folate to PABA is conveniently effected using a strongly acidic medium, eg 6M hydrochloric acid for 6 hours at 110° C. or by vapour phase hydrochloric acid hydrolysis in sealed containers (eg 1 hour at 150° C.), or 4N methanesulphonic acid. Desirably hydrolysis is effected using a metal catalyst, e.g a transition metal or compound thereof, in particular platinum or a platinum compound, in acidic solution, e.g. strong (eg ≧4M, preferably ≧5M, especially ≧6M) hydrochloric acid, optionally after an initial incubation with one or more protease.

In an alternative, highly preferred embodiment, acid hydrolysis of folate to PABA is effected under microwave irradiation. This can bring the time required for hydrolysis down by a factor of 5 or more, and possibly even more than 25.

In a further, highly preferred embodiment, oxidation with agents such as hydrogen peroxide, and/or treatment using a reducing agent such as sodium borohydride, can be employed to greatly increase the susceptibility of various folates to acidic hydrolysis by conversion of the various folates from the sample into acid sensitive derivatives. These derivatives are then rapidly degradable into PABA or PABA-glu. Preferably, a reduction and an oxidation method are used consecutively. Such a method may comprise, for example, treatment of a sample with sodium borohydride, followed by oxidation with hydrogen peroxide and potassium permanganate and finally lowering of the pH to around pH1, whereby to convert the various folates into acid susceptible folate derivatives and cause rapid hydrolysis of most, or most preferably all, of folate derivatives to a uniform PABA or PABA-glu product for analysis.

In addition, folates may be hydrolysed by oxidative photolysis, especially in the presence of photosensitising agents such as riboflavin. Such a method forms a further highly preferred embodiment and may employ visible, infra-red, or particularly ultra-violet light. The major products of such photolysis are typically PABA, PABA-glu and pteridine-6-carboxylic acid (PCA). Any of these products, and preferably any two or all three products may be determined as described above or by analogous methods, such as immunoassay of PCA.

Oxidative photolysis of folate may be performed by irradiating the sample with a high intensity light source, typically for >5 minutes at or around room temperature. The effectiveness of the photolysis depends principally upon the presence of dissolved oxygen, the irradiation time, the intensity of the light source and the temperature and pH in the solution. Certain additives like riboflavin work as photo sensitizers and dramatically increase the yield of the photolysis products. The use of such photosensitizers is thus highly preferred. Low pH (i.e. pH 1-5, especially pH 2-4, e.g. pH=3) is preferred, but is not essential. It can be carried out to completion even at a pH between 6-9. UV-light (e.g. wavelength around 350 nm or shorter) is preferred for rapid oxidation but is not essential since oxidation occurs quite rapidly, even when using visible wavelengths (e.g. >500 nm).

Oxidative photolysis can be performed directly on lysed samples or on samples pretreated with enzymes. The different forms of folate are all decomposed into PABA, PABA-glu and pteridine-6-carboxylic acid as the main products. Although all these species can be determined immunologically using antibodies with the necessary specificity, the detection of PABA or PABA-glu is preferred because the danger of cross-reactivity of the antibody against endogenous compounds is minimized. PABA and PABA-glutamic acid can be determined by using the fluorescent azo-technique described supra.

The acid hydrolysis may also be made more user-friendly by initial proteolysis, eg using enzymes such as pepsin, trypsin, chymotrypsin and carboxypeptidase A either alone or in combinations of two or more optionally together with further enzymes such as amylase and conjugase. The folate forms released can then be converted to PABA by acid hydrolysis as described above or by enzyme or metal catalysed degradations. This combination of techniques can result in liberation of PABA from folate-containing samples in a matter of minutes.

Any of the mild pteridine ring decomposition methods, or any combination thereof, which are described supra as providing PABA, may be simply modified to generate PABA-glu. Specifically, incubation of a blood or blood derived sample for a short period (e.g 10 min to 6 hours, preferably 30 min to 3 hours, most preferably 1-2 hours) at around physiological temperature will typically result in the removal of all but the terminal glutamate residue from the various folates by the action of naturally occurring conjugase(s). Alternatively, or in addition, an enzyme or enzyme cocktail comprising components such as conjugases, proteases and α-amylases may be used. In a preferred example, conjugases, proteases and α-amylases are used as a tri-enzyme mixture to accelerate the removal of the glutamate residues. Again these enzymes result in the removal of all but the final, terminal, glutamic acid residue.

By this method, followed by pteridine ring decomposition as described above, the various folates of a sample are converted to a single species (PABA-glu). As described supra, this uniform PABA-glu product may then be analysed by the methods used to detect PABA, or simple variants thereof. The formation and analysis of PABA-glu is thus equally effective in reflecting the total folate content as is the analysis of PABA formed under conditions which would also remove the glutamic acid residues.

Should the final glutamic acid be desirably removed under the mild conditions preferred in the present invention then the PABA-glu or its derivatives may be treated with an enzyme such as carboxy peptidase G2 under appropriate conditions. Alternatively, this or equivalent enzymes may be included in the enzymic treatment cocktail described for removal of the other glutamic acid residues, by which method, in combination with pteridine ring decomposition, PABA will be generated.

Following the acid hydrolysis to yield PABA or PABA-glu, if the binding partner is pH sensitive the pH of the sample may be adjusted (e.g. by addition of a base such as sodium hydroxide or a buffer), eg to a value closer to pH 7.

Where the binding partner is immobilized on a substrate, the sample is then brought into contact with the substrate and incubated to allow PABA:binding partner or PABA-glu:binding partner, binding to occur. Desirably the substrate is then rinsed to remove unbound material before the PABA:binding partner or PABA-glu:binding partner combination is assessed. In this format, it is preferred to use a second, labelled binding partner which binds either to the PABA/PABA-glu:binding partner complex or to the immobilized binding partner which has not bound to PABA/PABA-glu. Detection of the label thus gives a direct or indirect value for the PABA or PABA-glu concentration. The labels used may for example be fluorophores, chromophores, radiolabels, etc.

Where the binding partner is an aromatic amine or phenol, PABA is first converted to PDBA or PABA-glu to PDBA-glu, e.g. by reaction with sodium nitrite and hydrochloric acid. The PDBA or PDBA-glu containing sample is then contacted with the aromatic amine or phenol which may or may not be substrate bound. The aromatic amine or phenol must have an unsubstituted ring position ortho or para to the amino or hydroxy group but may be substituted at other ring positions or at the amine nitrogen and, as described below, such substituents may be used to select the desired colour or fluorescence wavelength of the azo compound formed by reaction with PDBA or PABA-glu as well as to couple the amine or phenol to a substrate. Where the amine or phenol is substrate bound, after incubation with the PDBA or PDBA-glu containing sample, the substrate may be rinsed to remove unbound material and the azo compound concentration can then be determined directly by spectrometric methods. Where the amine or phenol is not substrate bound, the concentration of the azo compound can again be determined directly by spectrometric methods. However as the sample will contain heme degradation products, in this case the amine or phenol is preferably selected so that the azo compound is fluorescent or has a characteristic absorption at a wavelength at which the “background” from the heme degradation products is relatively small.

Hydroxyl and amino groups, especially if they are ortho- or para- to the azo bond intensify the colour of azo compounds. This is exemplified by p-hydroxyazobenzene which has an absorption maximum at 349 nm (ethanol, ε=26300), and p-dimethylaminoazobenzene which has an absorption maximum at 408 nm (ethanol, ε=27540). Both azo-compounds absorb at longer wavelength and with higher molar absorptivity than azobenzene itself does.

Azo-compounds in which an electron donating substituent on one aromatic ring is conjugated with an electron-withdrawing group on the other ring have especially deep colours. A good example is provided by the group of azobenzenes where a nitro group is substituted on one ring ortho or para to the azo-linkage and a dialkylamino group is substituted on the other ring—also para to the azo linkage. This is because one resonance contributor of these compounds has a quinoid structure, a feature often associated with deep colour in azo compounds. An example of such a compound illustrating this characteristic is 4-dimethylamino-4′-nitrobenzene which has an absorption maximum at 478 nm with ε=33110 in ethanol.

Fluorescence may be expected-generally in molecules that are aromatic and contain multiple-conjugated double bonds with a high degree of resonance stability. Both classes of substances have delocalized n-electrons that can be placed in low-lying excited singlet states. One way of increasing the fluorescence of azo compounds is to prevent the excited single state from loosing the energy by intramolecular nonradiative transitions, i.e. transforming the excitation energy into vibrational movements within the molecule. Molecular rigidity lessens the possibility of competing nonradiative transitions by decreasing vibrations this minimizes intersystem crossing to the triplet state and collisional heat degradation. This is clearly illustrated by extending the alkyl chains of the tertiary amino group in 4-dialkylamino-4′-carboxyazobenzene. Many short alkyl chain length azobenzenes are practically non-fluorescent, while longer chain (≧C3) azobenzenes are fluorescent. Also, the fluorescence intensity increases with the alkyl chain length of the amino group. However, since this inevitably increases the hydrophobicity of the compounds, the decreased solubility of these long chain fluorescent azo-compounds may limit their use. Thus 4-di (C₃₋₆alkyl)amino-4′-carboxyazobenzenes are especially preferred.

Since reduced flexibility of the alkyl chain is a key element related to the fluorescence properties, one way of overcoming the solubility problem and further “immobilize” the alkyl chains is to use aromatic amines where the alkyl chains form stable ring structures. An example of such a compound is julolidine. This structure, incorporated in the azo-structure with the amine para to the azo bond, greatly reduces loss of excitation energy due to molecular vibration, resulting in increased fluorescence. Furthermore, adding extra phenyl rings in the azo-compounds, like those formed when naphthalene-derivatives (or higher order fused phenyl ring compounds) with amino and/or hydroxyl substituents (naphthol/naphthaleneamine) are reacted with diazonium group of PDBA or PDBA-glu, strengthens the fluorescence.

Generally, the most planar, rigid and sterically uncrowded molecule of a series of organic compounds is the most fluorescent one. The formation of chelates with metal ions in general also promotes fluorescence by promoting rigidity and minimizing internal vibrations. For this reason, hydroxyl or substituted or unsubstituted amino groups are preferably included in the amine or phenol at positions meta to the amine or hydroxyl groups. These will be capable of coordinating metal ions.

Such substituents strongly affect fluorescence. A substituent that delocalises the n-electrons, such as an —NH₂, —OH, —OCH₃, —F, —NHCH₃ or N(CH₃)₂ group, often enhances fluorescence, while electron-withdrawing substituents, e.g. —Cl, —Br, —I, —NHCOCH₃, —NO₂ or —COOH, decrease or completely quench the fluorescence.

Additionally, changes in the system pH may also influence fluorescence if it affects the charge status of the chromophore. Such changes can be explained by comparing resonance forms of anion and cation structures.

The substitution patterns and conjugated ring systems that enhance the conjugated system and/or lead to quinoid structures will generally give rise to bathochromic shifts resulting in absorption bands in the red part of the spectrum, possibly different from the absorption bands of the heme decomposition products. It is important to realise that the hydrolysis of hemeglobin will produce heme-decomposition products with absorption characteristics different from native heme, and the majority of these compounds will absorb in the blue part of the spectrum (and so appear light yellow-brownish in colour). This is obviously a beneficial situation since the spectral overlap between the heme derived chromophores and the diazo compound will be greatly reduced compared to the situation with native heme.

Where the binding partner is neither substrate bound nor an aromatic amine or a phenol, the PABA or PABA-glu containing sample is desirably first incubated with a labelled binding partner and then contacted with a second, substrate bound binding partner which is capable of binding either to the PABA/PABA-glu:binding partner complex or to the free binding partner. The substrate is then desirably rinsed to removed unbound material and the substrate-bound label is then detected giving a direct or indirect value for the PABA or PABA-glu concentration.

Alternatively, an unbound, fluorophore-labelled binding partner may be used with the concentration of PABA or PABA-glu being determined by the change in fluorescence polarization resulting from the formation of PABA/PABA-glu:binding partner complexes. Again however the fluorophore preferably has a characteristic emission wavelength at which heme degradation products provide minimal background.

Viewed from a further aspect, the invention provides a kit, for use in the performance of the assay of the invention, said kit comprising:
a) a folate hydrolysis reagent;
b) optionally an enzyme cocktail;
c) a PABA, PABA-glu, PDBA or PDBA-glu binding partner;
d) optionally a [PABA to PDBA or PABA-glu to PDBA-glu converting] reagent; and
e) optionally, a secondary binding partner.

The invention preferably provides a kit for use in the performance of the assay of the invention, said kit comprising:
a) a folate hydrolysis reagent;
b) a PABA or PDBA binding partner;
c) optionally a [PABA to PDBA converting] reagent; and
d) optionally, a secondary binding partner.

The assay method of the invention is preferably performed together with a homocysteine assay and/or a holo transcobalamin II assay, e.g. as described in U.S. Pat. No. 6,063,581, U.S. Pat. No. 5,631,127, WO 00/40973, WO 00/11479 and WO 00/17659.

The invention will now be described further with reference to the following non-limiting Examples:

### EXAMPLE 1

Conversion of Folate to PABA

A 200 μL sample of whole blood is mixed with 500 μL of 8.5M hydrochloric acid in borosilicate glass tubes with Teflon-lined screw caps and incubated for 6 hours at 110° C.

### EXAMPLE 2

Conversion of PABA to PDBA

The PABA containing composition of Example 1 is cooled to room temperature, diluted with 500 μL purified water and loaded onto a C18 solid phase extraction cartridge. The colourless eluant is diluted 1:10 v/v with water to produce a hydrogen chloride concentration of approx. 0.3M. Aqueous sodium nitrite solution (50 mg/ml) is added in a volume ratio of 2:1 (sample:NaNO₂ solution) and the mixture is allowed to react for ten minutes at 4° C.

### EXAMPLE 3

Conversion of PDBA to Diazo Compound

The PDBA solution of Example 2 is mixed with an aromatic amine or phenol solution (4 mg/mL in 10 mM aqueous phosphate buffer, 0.15M NaCl, pH 7.4) in a volume ratio of 3:2 (PDBA solution:amine/phenol solution) and allowed to react for 30 minutes. The diazo compound can then be detected spectrometrically.

In this Example, the following aromatic amines/phenols will typically be used: N,N-di-n-propyl-aminobenzene; N,N-di-n-butyl-aminobenzene; julolidine; phenol; and 2-hydroxynaphthalene.

### EXAMPLE 4

Combined Use of Hydrogen Peroxide and pH to Decompose all Folate Species into PABA-Monoglutamic Acid

1. Add 25 μl whole blood and lyse the cells using saponin (add to 100 mg/L final concentration) or ascorbic acid (added to 1% ascorbic acid, final concentration). Incubate for 2 hours at 37° C. to allow the conjugase present in the sample to remove all but the terminal glutamate residue.

2. Remove non-relevant molecules from whole blood using a molecular sieve (e.g. centrifuge concentrators) to leave fairly pure folate/binding protein complexes.

3. Dissociate folate from binding protein using 0.1% (final concentration) DTT and boil for 15 minutes or alternatively add DTT and 0.15N (final concentration) sodium hydroxide and incubate 2.5 minutes at 37° C.

4. Adjust pH to 6.0 and add sodium borohydride (6 mg/ml) and incubate at room temperature for 10 minutes to reduce folates.

5. Acidify using HCl to adjust pH and destroy the borohydride.

6. Adjust pH to 9.0 and add hydrogen peroxide and potassium permanganate (0.015% H₂O₂ and 0.1% KMNO₄, respectively) to oxidize, followed by excess hydrogen peroxide (0.3% H₂O₂) to precipitate the excess permanganate. The precipitated MnO₂ is removed by centrifugation 4000 g for 10 min.

7. Add catalase (0.1% catalase to a final concentration of ⅓ the volume of H₂O₂ present) to destroy the hydrogen peroxide.

8. Reduce pH to 1 with HCl and incubate for 2 hours at room temperature to drive the conversion of folate to PABA-glu.

9. Adjust the acidity of the sample to neutral pH and determine the amount of PABA-glu present either by using a specific antibody or by transforming PABA-glu into a fluorescent azo-dye as described e.g in Example 3. The amount of PABA-glu is determined from a standard curve and the resulting concentration reflects the total concentration of folate in the sample.

### EXAMPLE 5

Accelerated Removal of Non-Terminal Glutamates

As for Example 4, but in step 1, additional conjugases, like γ-Glu-X Carboxypeptidase; EC 3.4.19.9, or a trienzyme mixture composed of conjugase, protease and α-amylase, are added to help facilitate the release and transformation of folate and speed up the reactions.

### EXAMPLE 6

Combined Use of Hydrogen Peroxide and pH to Decompose all Folate Species into PABA

1. Step 1 as for Examples 4 and 5 but the proteolytic enzyme Carboxypeptidase G2 is used alone or in addition to the enzymes mentioned in Example 5. This further enzyme results in the removal of all glutamic acid residues from folate, resulting in the production of PABA rather than PABA-glu.

2. Remove non-relevant molecules from whole blood using a molecular sieve (e.g. centrifuge concentrators) to leave fairly pure folate/binding protein complexes.

3. Dissociate folate from binding protein using 0.1% (final concentration) DTT and boil for 15 minutes or alternatively add DTT and 0.15N (final concentration) sodium hydroxide and incubate 2.5 minutes at 37° C.

4. Adjust pH to 6.0 and add sodium borohydride (6 mg/ml) and incubate at room temperature for 10 minutes to reduce folates.

5. Acidify using HCl to adjust pH and destroy the borohydride.

6. Adjust pH to 9.0 and add hydrogen peroxide and potassium permanganate (0.015% H₂O₂) and 0.1% KMNO₄, respectively) to oxidize, followed by excess hydrogen peroxide (0.3% H₂O₂) to precipitate the excess permanganate. The precipitated MnO₂ is removed by centrifugation 4000 g for 10 min.

7. Add catalase (0.1% catalase to a final concentration of ⅓ the volume of H₂O₂ present) to destroy the hydrogen peroxide.

8. Reduce pH to 1 with HCl and incubate for 2 hours at room temperature to drive the conversion of folate to PABA.

9. Determine the amount of PABA present either by immunodetection using a specific antibody (after first adjusting the pH to an appropriate pH optimal for the immunoreaction, e.g. to a pH between 7.0 and 8.5) or alternatively by transforming PABA into a fluorescent azo-dye as described herein e.g. in Example 3. The amount of PABA is determined from a standard curve and the resulting concentration reflects the total concentration of folate in the sample.

### EXAMPLE 7

Use of Oxidative, Photolysis of Folate Species into PABA Photolysis, but Without the Use of Endogenous Enzymes

1. Lyse red cells using saponin (add to 100 mg/L final concentration) or ascorbic acid (added to 1% ascorbic acid, final concentration) and incubate for 1 hours at 37° C.

2. Identical to step 2, Example 4.

3. Identical to step 3, Example 4.

4. Irradiate the sample with a UV-light source for 10 minutes at room temperature. The main photolysis products are PABA, PABA-glu and pteridine-6-carboxylic acid which all reflect the initial total concentration of folate in the sample.

5. Determine the amount of PABA or PABA-glu present either by immunodetection using a specific antibody (after first adjusting the pH to an appropriate pH optimal for the immunoreaction, e.g. between 7.0 and 8.5) or alternatively by transforming PABA or PABA-glu into a fluorescent azo-dye as described in Example 3. The amount of PABA or PABA-glu in the sample is determined from a standard curve made with appropriate standards covering the concentration range of folate in blood samples.

1. A method of assaying for folate in a folate containing sample, wherein at least some of said folate comprises at least one attached glutamate residue, said method comprising:
subjecting said sample to hydrolysis to release paraminobenzoic acid, p-aminobenzoyl glutamic acid, or a salt thereof, contacting the released paraminobenzoic acid, p-aminobenzoyl glutamic acid, salt, or a diazo derivative thereof, with a binding partner therefor; and directly or indirectly detecting the resulting binding partner: paraminobenzoic acid, binding partner: p-aminobenzoyl glutamic acid, or salt or derivative combination wherein said method does not comprise any chromatographic separation steps.

2. A method as claimed in claim 1 wherein said sample is a blood derived sample.

3. A method as claimed in claim 1 wherein said binding partner is selected from an antibody, an antibody fragment, a single chain antibody, a single chain antibody fragment, an oligopeptide, anoligonucleotide and a small organic molecule.

4. A method as claimed in claim 3 wherein said small organic molecule is an aromatic tertiary amine, phenol or phenol derivative capable of forming a diazo compound with paradiazobenzoic acid (PDBA) or paradiazobenzoyl glutamate (PDBA-glu).

5. A method as claimed in claim 1 wherein said hydrolysis comprises treating said sample with a metal catalyst under acidic conditions.

6. A method as claimed in claim 1 wherein said hydrolysis comprises treating said sample with microwave radiation.

7. A method as claimed in claim 1 wherein said hydrolysis comprises treatment with an oxidising agent.

8. A method as claimed in claim 7 wherein the oxidising agent is hydrogen peroxide and/or potassium permanganate.

9. A method as claimed in claim 1 wherein said hydrolysis comprises treatment with a reducing agent.

10. A method as claimed in claim 9 wherein the reducing agent is sodium borohydride.

11. A method as claimed in claim 1 wherein said hydrolysis comprises oxidative photolysis.

12. A method as claimed in claim 11 wherein said oxidative photolysis is carried out in the presence of a photosensitiser.

13. A method as claimed in claim 1 wherein said sample is incubated in the presence of naturally occurring and/or added enzymes whereby to remove all but the terminal glutamate residue from said folate and wherein the product of the hydrolysis is PABA-glu.

14. A method as claimed in claim 1 wherein said sample is incubated in the presence of at least one added enzyme, whereby to remove all glutamate residues from said folate, and wherein the product of the hydrolysis is PABA.

15. A method as claimed in claim 1 wherein said binding partner: paraminobenzoic acid, binding partner: p-aminobenzoyl glutamic acid, or salt or derivative combination is detected directly by absorbence or fluorescence.

16. A method as claimed in claim 1 wherein said binding partner: paraminobenzoic acid, binding partner: p-aminobenzoyl glutamic acid, or salt or derivative combination is detected indirectly by means of a secondary binding partner.

17. A kit for use in the performance of the assay of the invention, said kit comprising:
i) a folate hydrolysis reagent; and
ii) a PABA, PABA-glu, PDBA or PDBA-glu binding partner.

18. A kit as claimed in claim 17 additionally comprising an enzyme or enzyme cocktail.

19. A kit as claimed in claim 17 additionally comprising a PABA to PDBA or PABA-glu to PDBA-glu converting reagent.

20. A kit as claimed in claim 17 additionally comprising a secondary binding partner.
